(19) United States
(12) Patent Application Publication
(10) Pub. No.: US 2016/0081663 A1
(43) Pub. Date: Mar. 24, 2016

(54) METHOD AND SYSTEM FOR AUTOMATED DETECTION AND MEASUREMENT OF A TARGET STRUCTURE

(71) Applicant: General Electric Company, Schenectady, NY (US)

(72) Inventors: Jixu Chen, Niskayuna, NY (US); Kajoli Banerjee Krishnan, Bangalore (IN)

(21) Appl. No.: 14/489,497

(22) Filed: Sep. 18, 2014

(51) Int. Cl. A61B 8/08; A61B 5/00; A61B 8/00; G06T 7/00

(52) U.S. Cl. CPC A61B 8/5223; G06T 7/0014; G06T 7/0085; G06T 7/0089; A61B 8/483; A61B 5/0073; A61B 8/0866; A61B 8/4245; G06T 2207/10136; G06T 2207/20124; G06T 2207/30044; A61B 2576/00

(57) ABSTRACT

A system and method for imaging a subject are disclosed. A plurality of edge points corresponding to a set of candidate structures are determined in each image frame in a plurality of 3D image frames corresponding to a volume in the subject. A target structure is detected from the set of candidate structures by applying constrained shape fitting to the edge points in each image frame. A subgroup of image frames including the target structure is identified from the 3D frames. A subset of edge points corresponding to the target structure is determined in each of the subgroup of image frames. A plurality of 2D scan planes corresponding to the subset of edge points is determined, and ranked using a determined ranking function to identify a desired scan plane. A diagnostic parameter corresponding to the target structure is measured using a selected image frame that includes the desired scan plane.

### BACKGROUND

Embodiments of the present specification relate generally to diagnostic imaging, and more particularly to a method and system for automatically detecting and measuring a target structure in an ultrasound image.

Medical diagnostic ultrasound is an imaging modality that employs ultrasound waves to probe the acoustic properties of biological tissues and produces corresponding images. Particularly, ultrasound systems are used to provide an accurate visualization of muscles, tendons, and other internal organs to assess their size, structure, and any pathological conditions using near real-time images. For example, ultrasound images have been extensively used in prenatal imaging for assessing gestational age (GA) and weight of a fetus. In particular, two-dimensional (2D) and/or three-dimensional (3D) ultrasound images are employed for measuring desired features of the fetal anatomy such as the head, abdomen, and/or femur. Measurement of the desired features, in turn, allows for determination of the GA, assessment of growth patterns, and/or identification of anomalies in the fetus.

By way of example, accurate measurement of the biparietal diameter (BPD) and/or head circumference (HC) of the fetus in the second and third trimesters of pregnancy provides an accurate indication of fetal growth and/or weight. Typically, accurate measurement of the HC and/or BPD entails using a clinically prescribed 2D scan plane identified from a 3D volume for the measurements. In common clinical practice, a radiologist attempts to select the clinically prescribed scan plane by repeatedly repositioning a transducer probe over an abdomen of the patient. In the clinically prescribed scan plane, the fetal head is visualized in an ultrasound image that includes a cavum septum pellucidum, thalami, and choroid plexus in the atrium of lateral ventricles such that the cavum septum pellucidum appears as an empty box and the thalami resemble a butterfly. Accurate BPD and HC measurements using the clinically prescribed scan plane allows for accurate fetal weight and/or size estimation, which in turn, aids in efficient diagnosis and prescription of treatment for the patient.

Acquisition of an optimal image frame that includes the clinically prescribed scan plane for satisfying prescribed clinical guidelines, however, may be complicated. For example, acquisition of the optimal image frame may be impaired due to imaging artifacts caused by shadowing effect of bones, near field haze resulting from subcutaneous fat layers, unpredictable patient movement, and/or ubiquitous speckle noise. Additionally, an unfavorable fetal position, fetus orientation, and/or change in shape of the fetal head due to changes in the transducer pressure may also confound the BPD and HC measurements.

Moreover, operator and/or system variability may also limit reproducibility of the BPD and HC measurements. For example, when using an ultrasound system that includes a low cost position sensor having limited range, accuracy of biometric measurements may vary significantly based on a selection of a reconstruction algorithm and/or skill of an operator. Additionally, sub-optimal ultrasound image settings such as gain compensation and dynamic range may impede an ability to visualize internal structures of the human body. Furthermore, even small changes in positioning the ultrasound transducer may lead to significant changes in the visualized image frame, thus leading to incorrect measurements.

Accurate ultrasound measurements, thus, typically entail meticulous attention to detail. While experienced radiologists may be able to obtain accurate measurements with less effort and time, acquiring clinically acceptable biometric measurements typically requires much greater effort and time from inexperienced users and/or entails use of expensive 3D ultrasound probes. Accordingly, accuracy of conventional ultrasound imaging methods may depend significantly upon availability of state-of-the-art ultrasound probes and/or skill and experience of the radiologist, thereby limiting availability of quality imaging services, for example, to large hospitals and urban areas. Scarcity of skilled and/or experienced radiologists in remote or rural regions, thus, may cause these regions to be poorly or under-served.

Accordingly, certain conventional ultrasound imaging methods have been known to employ training algorithms and/or semi-automated methods that use image-derived characteristics to assist in diagnosis and treatment. These conventional methods typically rely on the radiologist's selection of the optimal image frame from a plurality of image frames. In a conventional clinical workflow, the radiologist may continue to search for a better image frame even after identifying an acceptable image frame in the hope of obtaining measurements that are more accurate. However, upon failing to find a better image frame, the radiologist may have to manually scroll back to an originally acceptable image frame, thus prolonging imaging time and hindering reproducibility. Ultrasound imaging using conventional methods, cost-effective ultrasound scanners, and/or by a novice radiologist, therefore, may not allow for measurements suited for real-time diagnosis and treatment.

### BRIEF DESCRIPTION

In accordance with certain aspects of the present specification, a system for imaging a subject is presented. The system includes an acquisition subsystem configured to obtain a plurality of three-dimensional image frames corresponding to a volume of interest in the subject. The system also includes a processing unit in operative association with the acquisition subsystem and configured to determine a plurality of edge points corresponding to a set of candidate structures in each image frame in the plurality of three-dimensional image frames. Further, the processing unit is configured to identify a target structure from the set of candidate structures by applying constrained shape fitting to the plurality of edge points in each image frame in the plurality of three-dimensional image frames. Additionally, the processing unit is configured to identify a subgroup of image frames from the plurality of three-dimensional image frames, where each image frame in the subgroup of image frames comprises the target structure. Moreover, the processing unit is configured to determine a subset of edge points corresponding to the target structure from the plurality of edge points in each image frame in the subgroup of image frames. Further, the processing unit is configured to determine a plurality of two-dimensional candidate scan planes corresponding to the subset of edge points in each image frame in the subgroup of image frames. Additionally, the processing unit is configured to rank the plurality of two-dimensional candidate scan planes corresponding to each image frame in the subgroup of image frames using a determined ranking function. The processing unit is configured to identify a desired scan plane from the plurality of two-dimensional candidate scan planes based on the ranking. Furthermore, the processing unit is configured to measure a diagnostic parameter corresponding to the target structure using a selected image frame in the plurality of three-dimensional image frames, where the selected image frame comprises the desired scan plane.

In accordance with certain further aspects of the present specification, a method for ultrasound imaging of a subject is disclosed. The method includes determining a plurality of edge points corresponding to a set of candidate structures in each image frame in a plurality of three-dimensional image frames corresponding to a volume of interest in the subject. Additionally, the method includes detecting a target structure from the set of candidate structures by applying constrained shape fitting to the plurality of edge points in each image frame in the plurality of three-dimensional image frames. Further, the method includes identifying a subgroup of image frames from the plurality of three-dimensional image frames, where each image frame in the subgroup of image frames comprises the target structure. The method also includes determining a subset of edge points corresponding to the target structure from the plurality of edge points in each image frame in the subgroup of image frames. Moreover, the method includes determining a plurality of two-dimensional candidate scan planes corresponding to the subset of edge points in each image frame in the subgroup of image frames. Additionally, the method includes ranking the plurality of two-dimensional candidate scan planes corresponding to each image frame in the subgroup of image frames using a determined ranking function. Further, the method includes identifying a desired scan plane from the plurality of two-dimensional candidate scan planes based on the ranking. The method also includes measuring a diagnostic parameter corresponding to the target structure using a selected image frame in the subgroup of image frames, where the selected image frame comprises the desired scan plane. Additionally, a non-transitory computer readable medium that stores instructions executable by one or more processors to perform the method for imaging a subject is also presented.

### DRAWINGS

These and other features and aspects of embodiments of the present specification will become better understood when the following detailed description is read with reference to the accompanying drawings in which like characters represent like parts throughout the drawings, wherein:

FIG. 1 is a schematic representation of an exemplary ultrasound imaging system, in accordance with aspects of the present specification;

FIG. 2 is a flow chart illustrating an exemplary method for ultrasound imaging, in accordance with aspects of the present specification;

FIG. 3 is a flow chart illustrating an exemplary method for detecting a target structure using a constrained shape fitting method, in accordance with aspects of the present specification;

FIG. 4 is a graphical representation of fitting scores computed for a plurality of ellipsoids fit to candidate structures in a plurality of image frames, in accordance with aspects of the present specification;

FIG. 5 is a graphical representation of fitting scores computed for a plurality of ellipsoids fit to candidate structures in a plurality of image frames corresponding to different regions in a fetus, in accordance with aspects of the present specification;

FIG. 6 is a flow chart illustrating an exemplary method for ranking candidate scan planes corresponding to the target structure, in accordance with aspects of the present specification; and

FIG. 7 is a diagrammatical representation of a plurality of image frames corresponding to a volume of interest in a subject, in accordance with aspects of the present specification.

### DETAILED DESCRIPTION

The following description presents systems and methods for automatically detecting and measuring a target structure in an ultrasound image. Particularly, certain embodiments presented herein describe the systems and methods configured to accurately detect one or more target structures in a plurality of image frames and identify an optimal image frame that includes the one or more target structures in a desired scan plane. As used herein, the term “desired scan plane” may be used to refer to a cross-sectional slice of an anatomical region that satisfies clinical, user-defined, and/or application-specific guidelines to provide accurate and reproducible measurement of a target structure. Furthermore, the term “optimal image frame” is used to refer to an image frame that includes the target structure in the desired scan plane that satisfies the prescribed guidelines for providing one or more desired measurements of the target structure.

Particularly, the target structure, for example, may include one or more anatomical regions and/or features such as a head, an abdomen, a spine, a femur, the heart, veins, and arteries corresponding to a fetus, and/or an interventional device such as a catheter positioned within the body of a patient. In accordance with aspects of the present specification, the target structures may be detected in the plurality of image frames using a constrained shape fitting method. Additionally, candidate scan planes corresponding to the detected target structures may be identified and ranked using a boosted ranking function so as to identify the desired scan plane. Moreover, an image frame that includes the desired scan plane may be identified as an optimal image frame. Embodiments of the present systems and methods may then be used to automatically measure a desired biometric parameter corresponding to the target structure detected in the optimal image frame.

In accordance with further aspects of the present specification, embodiments of the present systems and methods may also allow for communication of a rank or a quality indicator corresponding to the image frames to a user for use in identifying the optimal image frame. The quality indicator may be representative of a probability of each of the image frames generated in real-time to provide biometric measurements of the target structures that satisfy clinical, user-defined, and/or application-specific guidelines. Additionally, the quality indicator may be representative of a relative distance or difference between a scan plane corresponding to a current image frame and the desired scan plane. The quality indicator, thus, may also be used for guiding one or more subsequent data acquisitions.

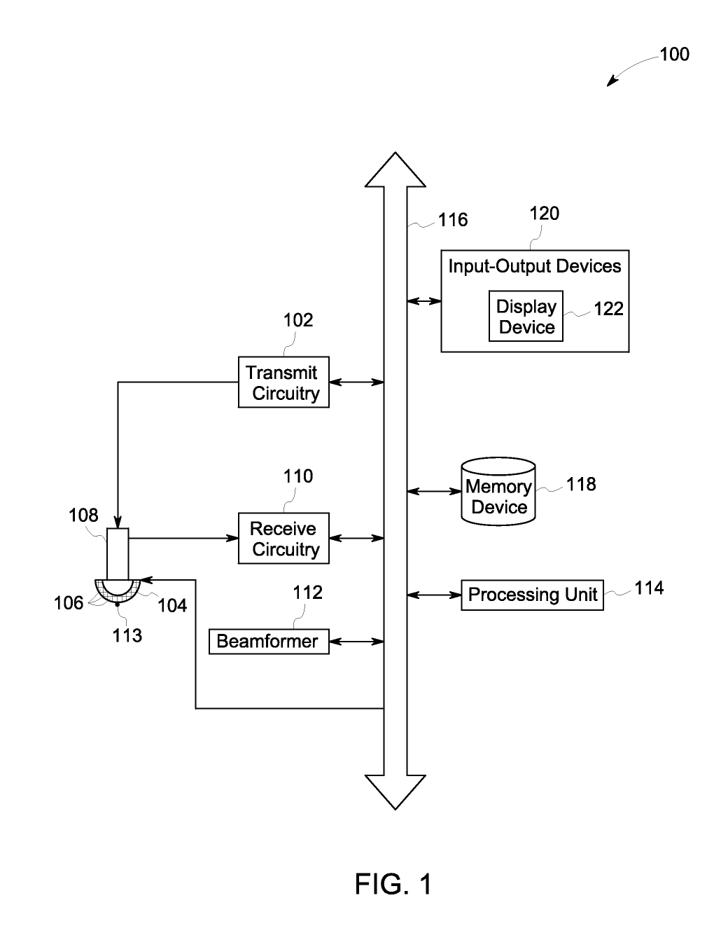

Although the following description includes embodiments relating to medical diagnostic ultrasound imaging, these embodiments may be adapted for implementation in other medical imaging systems. The other systems, for example, may include optical imaging systems and/or systems that monitor targeted drug and gene delivery. In certain embodiments, the present systems and methods may also be used for non-medical imaging, for example, during nondestructive evaluation of materials that may be suitable for ultrasound imaging and/or for security screening. An exemplary environment that is suitable for practising various implementations of the present system will be described in the following sections with reference to FIG. 1.

FIG. 1 illustrates an exemplary ultrasound system 100 for automatically detecting and measuring a target structure in an ultrasound image. To that end, the system 100 may be configured as a console system or a cart-based system. Alternatively, the system 100 may be configured as a portable and/or battery-operated system, such as a hand-held, laptop-based, and/or smartphone-based imaging system. Particularly, implementing the system 100 as a portable system may aid in extending availability of high quality ultrasound imaging facilities to rural regions where skilled and experienced radiologists are typically in short supply.

In one embodiment, the system 100 may be configured to automatically detect a target structure in an image frame. Additionally, the system 100 may be configured to automatically identify an optimal image frame that includes the target structure in a desired scan plane from a plurality of image frames. Particularly, the system 100 may be configured to detect the target structure and identify a corresponding desired scan plane using a constrained shape fitting method and a determined ranking function, respectively. The image frame including the desired scan plane may then be used to obtain accurate biometric measurements that are indicative of one or more characteristics features or a current condition of the subject.

For clarity, the present specification is described with reference to automatically detecting a head of a fetus and identifying an optimal image frame for accurately measuring a corresponding biparietal diameter (BPD) and/or a head circumference (HC). However, certain embodiments of the present specification may allow for automatic detection and identification of optimal image frames for measuring other target structures such as the femur or aorta corresponding to the fetus. Additionally, embodiments of the present specification may also be employed for real-time detection and/or measurement of other biological structures, and/or non-biological structures such as manufactured parts, catheters, or other surgical devices visualized in the plurality of image frames.

In one embodiment, the system 100 may be configured to acquire a plurality of three-dimensional (3D) image frames corresponding to a volume of interest (VOI) in the subject. The 3D image frames allow for extraction of a plurality of scan planes that provide different views of the target structure. For example, when imaging the subject such as a fetus, the 3D image frames provide different scan planes for optimal visualization of the fetal heart, the hepatic vein, the placenta previa, presence of twin babies, and the like that may not be readily obtained using 2D ultrasound images.

In certain embodiments, the system 100 may include transmit circuitry 102 configured to drive an array 104 of the transducer elements 106 housed within a transducer probe 108 for imaging the subject. Specifically, the transmit circuitry 102 may be configured to drive the array 104 of transducer elements 106 to emit ultrasonic pulses into a body or the VOI of the subject. At least a portion of these ultrasonic pulses back-scatter from the VOI to produce echoes that return to the transducer array 104 and are received by receive circuitry 110. In one embodiment, the receive circuitry 110 may be operatively coupled to a beamformer 112 that may be configured to process the received echoes and output corresponding radio frequency (RF) signals.

In certain embodiments, the system 100 may further include a position sensor 113 disposed proximal one or more surfaces of the transducer probe 108 to measure a corresponding position and/or orientation. The position sensor 113, for example, may include acoustic, inertial, electromagnetic, radiofrequency identification (RFID), magnetoresistance-based, and/or optical sensing devices. In one embodiment, the position sensor 113 may be mounted on an outer surface of the transducer probe 108 for tracking a position and/or orientation of a tip of the transducer probe 108. In an alternative embodiment, however, the position sensor 113 may be integrated within the transducer probe 108. Specifically, the position sensor 113 may be disposed outside or within a housing of the transducer probe 108 to allow use of conventional freehand scanning techniques such as articulated arms, acoustic sensing, magnetic field sensing, and/or image-based sensing.

In accordance with aspects of the present specification, while the transducer 104 acquires image information corresponding to the target structure, the position sensor 113 may be configured to determine position and/or orientation information corresponding to the transducer probe 108. For example, when using a magnetoresistance-based position sensor 113, the position sensor 113 may be configured to continually detect a change in a strength and/or orientation of a designated magnetic field during the movement of the transducer probe 108. The detected changes in the magnetic field, in turn, may be used to determine changes in a position and/or orientation of the transducer probe 108 relative to a reference position and/or orientation. In one embodiment, the position and/or orientation information (hereinafter referred to as “position information”) may then be used in conjunction with the echoes received by the receive circuitry 110 to reconstruct the 3D image of the target structure. Specifically, use of the position sensor 113 during freehand scanning may aid in acquisition of arbitrary volumes by allowing for a greater degree of translation and rotation of the ultrasound probe 108. Additionally, use of the relatively inexpensive position sensor 113 combined with efficient image reconstruction may obviate a need for use of expensive 3D ultrasound imaging components in the system 100.

Although FIG. 1 illustrates the position sensor 113, the transducer array 104, the transmit circuitry 102, the receive circuitry 110, and the beamformer 112 as distinct elements, in certain embodiments, two or more of these elements may be implemented together as an independent acquisition subsystem in the system 100. Such an acquisition subsystem may similarly be configured to acquire image data corresponding to the subject such as the patient or the fetus in addition to position information corresponding to the transducer probe 108 for use in generating a 3D image of the target structure and determining corresponding biometric measurements.

Further, in certain embodiments, the system 100 may include a processing unit 114 configured to receive and process the acquired image data and position information in accordance with a plurality of selectable ultrasound imaging modes. Particularly, the processing unit 114 may be configured to receive and process the acquired image data and the position information in near real-time and/or in an offline mode to reconstruct a 3D image of the target structure. Accordingly, in one embodiment, the processing unit 114 may be operatively coupled to the position sensor 113, the beamformer 112, the transducer probe 108, and/or the receive circuitry 110.

In one embodiment, the processing unit 114 may be configured to provide control and timing signals through a communications link 116 to different components of the system 100. Accordingly, the processing unit 114 may include devices such as one or more general-purpose or application-specific processors, digital signal processors, microcomputers, microcontrollers, Application Specific Integrated Circuits (ASICs), Field Programmable Gate Arrays (FPGA), or other suitable devices in communication with other components of the system 100.

Additionally, in one embodiment, the processing unit 114 may be configured to store the control and timing signals, position information, acquired image data, and other suitable information such as clinical protocols, operator input, and/or patient data in a memory device 118. The memory device 118 may include storage devices such as a random access memory, a read only memory, a disc drive, solid-state memory device, and/or a flash memory. In one embodiment, the processing unit 114 may be configured to use the stored information for configuring the transducer elements 106 to direct one or more groups of pulse sequences toward the VOI corresponding to the subject such as the fetus.

Moreover, in certain embodiments, the processing unit 114 may be configured to use the stored information for tracking displacements in the VOI caused in response to the incident pulses to determine one or more characteristics of underlying tissues. These characteristics, for example, may include size of a head, abdomen, or femur of the fetus that aid determination of gestational age (GA), assessment of growth patterns, and identification of anomalies in the fetus. The displacements and characteristics, thus determined, may be stored in the memory device 118. Additionally, the displacements and/or the determined characteristics may be communicated to a user, such as a radiologist, for further assessment.

Furthermore, in certain embodiments, the processing unit 114 may also be coupled to one or more user input-output devices 120 for receiving commands and inputs from the user. The input-output devices 120, for example, may include devices such as a keyboard, a touchscreen, a microphone, a mouse, a control panel, a display device 122, a foot switch, a hand switch, and/or a button. In one embodiment, the display device 122 may include a graphical user interface (GUI) for providing the user with configurable options for imaging desired regions of the subject. By way of example, the configurable options may include a selectable image frame, a selectable region of interest (ROI), a desired scan plane, a delay profile, a designated pulse sequence, a desired pulse repetition frequency, and/or other suitable system settings to image the desired ROI. Additionally, the configurable options may further include a choice of diagnostic information to be communicated to the user. The diagnostic information, for example, may include a HC, BPD, length of a femur, and/or an abdominal circumference of the fetus. Additionally, the diagnostic information may also be estimated from the signals received from the subject in response to the ultrasound pulses and/or the position information determined based on measurements acquired by the position sensor 113.

In accordance with certain further aspects of the present specification, the processing unit 114 may be configured to process the received signals and the position information to generate 3D image frames and/or the requested diagnostic information. Particularly, in one embodiment, the processing unit 114 may be configured to continually register acquired 3D image data sets to the position information, thereby allowing for determining accurate geometrical information corresponding to the resulting ultrasound images. Particularly, the processing unit 114 may be configured to process the RF signal data in conjunction with the corresponding position information to generate 2D, 3D, and/or four-dimensional (4D) images corresponding to the target structure. Additionally, in certain embodiments, the processing unit 114 may be configured to digitize the received signals and output a digital video stream on the display device 122. Particularly, in one embodiment, the processing unit 114 may be configured to display the video stream on the display device 122 along with patient-specific diagnostic and/or therapeutic information in real-time while the patient is being imaged.

Similarly, the processing unit 114 may also be configured to generate and display the 3D image frames in real-time while scanning the VOI and receiving corresponding echo signals. As used herein, the term “real-time” may be used to refer to an imaging rate of at least 30 image frames per second (fps) with a delay of less than 1 second. Additionally, in one embodiment, the processing unit 114 may be configured to customize the delay in reconstructing and rendering the image frames based on system-specific and/or imaging requirements. For example, the processing unit 114 may be configured to process the RF signal data such that a resulting image is rendered at the rate of 20 fps on the associated display device 122 that is communicatively coupled to the processing unit 114.

In one embodiment, the display device 122 may be a local device. Alternatively, the display device 122 may be suitably located to allow a remotely located medical practitioner to assess diagnostic information corresponding to the subject. In certain embodiments, the processing unit 114 may be configured to update the image frames on the display device 122 in an offline and/or delayed update mode. Particularly, the image frames may be updated in the offline mode based on the echoes received over a determined period of time. However, in certain embodiments, the processing unit 114 may also be configured to dynamically update and sequentially display the updated image frames on the display device 122 as and when additional frames of ultrasound data are acquired.

As previously noted, the processing unit 114 may be configured to automatically detect the target structure and identify an optimal 2D image frame from a 3D image volume. Particularly, the optimal 2D image frame may depict the target structure in a desired 2D scan plane that allows for accurate biometric measurements. For example, for obtaining clinically acceptable BPD and HC measurements, the desired scan plane is representative of a scan plane that visualizes the cavum septum pellucidum, thalami, and choroid plexus in the atrium of lateral ventricles such that the cavum septum pellucidum appears as an empty box and the thalami resemble a butterfly. However, in a conventional ultrasound imaging system, obtaining an optimal image frame that includes the desired scan plane for accurate BPD and HC measurements is a challenging and time consuming procedure.

Embodiments of the present specification allow for automatic detection of the target structure such as a fetal head in a subgroup of the acquired image frames by employing a constrained shape fitting method. Particularly, in one embodiment, the processing unit 114 may be configured to implement a constrained ellipsoid fitting method that fits different ellipsoids to selected candidate structures in an image frame for detecting the fetal head. Additionally, the processing unit 114 may be configured to apply certain constraints to the ellipsoid fitting method based on a known geometry of the fetal head. The constraints, for example, may include a condition that a selected candidate structure in an image frame may be identified as the fetal head only if long and short axes of an ellipsoid corresponding to the selected candidate structure are substantially similar.

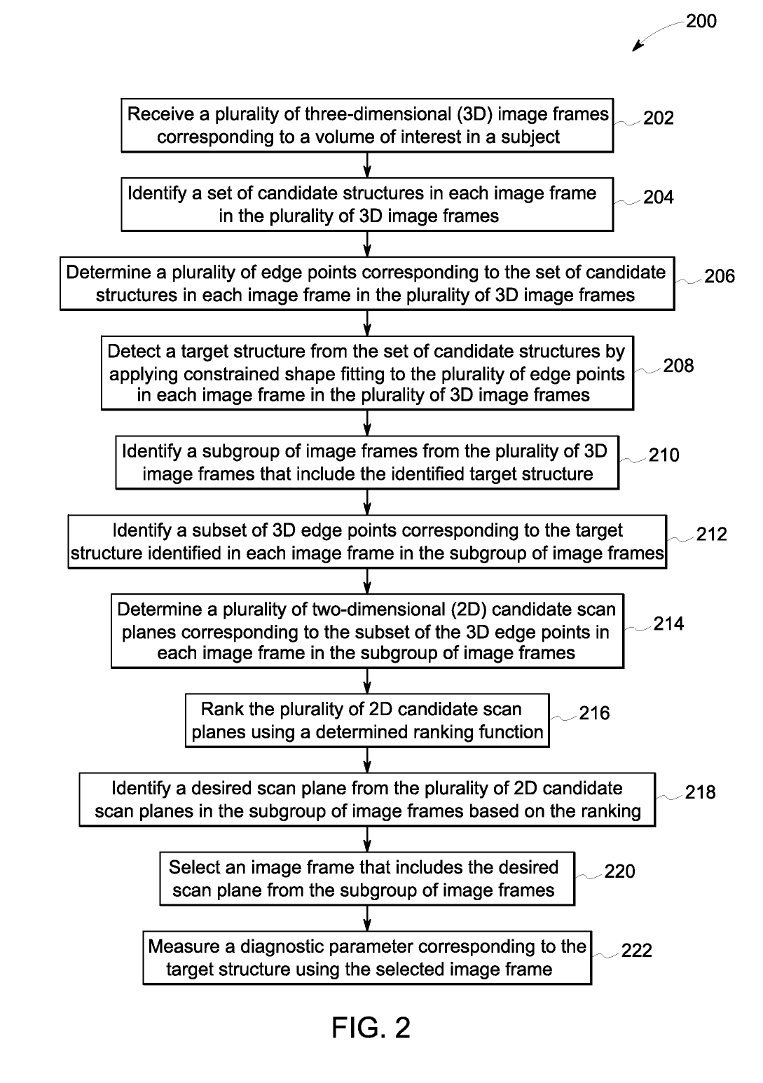

Further, the processing unit 114 may be configured to compute a fitting score for each of the ellipsoids fit to the selected candidate structures in the image frame. In one embodiment, an ellipsoid that satisfies the designated constraints and has the highest fitting score may be identified as the fetal head in the image frame. Moreover, a subgroup of the image frames that includes the fetal head may be identified. An exemplary embodiment of the ellipsoid fitting method for detecting the fetal head will be described in greater detail with reference to FIGS. 2-5.

Additionally, the processing unit 114 may be configured to identify a plurality of 2D candidate scan planes that correspond to the fetal head detected in each image frame in the subgroup of image frames. Further, the processing unit 114 may be configured to rank the 2D candidate scan planes using a determined ranking function. In one embodiment, the determined ranking function corresponds to a boosted ranking function. Particularly, the processing unit 114 may be configured to implement the boosted ranking function by extracting a set of representative image features from the 2D candidate scan planes, for example, using maximum rejection projection (MRP). Use of MRP allows projection of the candidate scan planes to a lower dimensional space where the desired scan plane is easily distinguishable from clinically unsuitable scan planes.

Additionally, training data including image pairs that are previously ranked by a skilled radiologist may be used to train a determined ranking function corresponding to the extracted set of representative image features. In one embodiment, the ranking function may rank the candidate scan planes, for example, using an iterative gradient descent method. Particularly, the ranking function uses the gradient ascent method to identify the highest or lowest ranked scan plane from the candidate scan planes as the desired scan plane.

In certain embodiments, the determined ranks may correspond to a quality indicator that indicates a suitability of a corresponding image frame for obtaining one or more desired biometric measurements. Alternatively, the ranks may be indicative of a difference between a particular image frame and the optimal image frame. In such embodiments, the processing unit 114 may be configured to communicate the rank of each image frame to a user in a visual form using the display device 122. For example, the ranks of the different image frames may be represented visually on the display device 122 using a color bar, a pie chart, and/or a number. Alternatively, the processing unit 114 may be configured to communicate the ranks of the different image frames using an audio and/or a video feedback. The audio feedback, for example, may include one or more beeps or speech in a selected language.

Moreover, in one embodiment, the audio and/or video feedback may include information representative of recommended remedial actions in case the candidate scan planes differ substantially from the desired scan plane. Alternatively, the processing unit 114 may be configured to transmit control signals to the system 100 to reinitiate scanning of the VOI, automatically or based on user input, if the ranks corresponding to the image frames are less than a clinically acceptable threshold. In certain embodiments, the processing unit 114 may be configured to ‘auto-freeze’ the image frame having the highest rank. Additionally, the processing unit 114 may be configured to trigger automated measurements corresponding to the target structure once the optimal image frame including the desired scan plane is identified.

Such real-time detection and/or measurement of the biometric parameters eliminates subjective and time-consuming manual assessment of image frames by a radiologist for identifying the optimal image frame suitable for obtaining clinically acceptable biometric measurements. Further, use of the constrained shape fitting and boosted ranking functions allows for greater accuracy in real-time detection of the target structure and corresponding biometric measurements. Embodiments of the present specification, thus, provide accuracy, repeatability, and reproducibility in biometry measurements, thereby resulting in consistent imaging performance even when performed by novice radiologists or different imaging systems. An exemplary method for automatically detecting and measuring a target structure in an image frame will be described in greater detail with reference to FIG. 2.

FIG. 2 illustrates a flow chart 200 depicting an exemplary method for ultrasound imaging. In the present specification, embodiments of the exemplary method may be described in a general context of computer executable instructions on a computing system or a processor. Generally, computer executable instructions may include routines, programs, objects, components, data structures, procedures, modules, functions, and the like that perform particular functions or implement particular abstract data types.

Additionally, embodiments of the exemplary method may also be practised in a distributed computing environment where optimization functions are performed by remote processing devices that are linked through a wired and/or wireless communication network. In the distributed computing environment, the computer executable instructions may be located in both local and remote computer storage media, including memory storage devices.

Further, in FIG. 2, the exemplary method is illustrated as a collection of blocks in a logical flow chart, which represents operations that may be implemented in hardware, software, or combinations thereof. The various operations are depicted in the blocks to illustrate the functions that are performed, for example, during steps of applying constrained shape fitting, ranking a plurality of 2D candidate scan planes, and/or measuring a biometric parameter corresponding to the target structure in the exemplary method. In the context of software, the blocks represent computer instructions that, when executed by one or more processing subsystems, perform the recited operations.

The order in which the exemplary method is described is not intended to be construed as a limitation, and any number of the described blocks may be combined in any order to implement the exemplary method disclosed herein, or an equivalent alternative method. Additionally, certain blocks may be deleted from the exemplary method or augmented by additional blocks with added functionality without departing from the spirit and scope of the subject matter described herein. For discussion purposes, the exemplary method will be described with reference to the elements of FIG. 1.

Embodiments of the present specification allow for automatic detection and measurement of a target structure and identification of an optimal image frame that includes the target structure in a desired scan plane. As previously noted, the desired scan plane may correspond to a cross-sectional slice of a VOI that satisfies clinical, user-defined, and/or application-specific guidelines to provide accurate and reproducible measurements of the target structure. The target structure, for example, may include one or more anatomical regions and/or features such as a head, an abdomen, a spine, a femur, the heart, veins, and arteries corresponding to a fetus, and/or an interventional device such as a catheter positioned within the body of a patient.

Particularly, certain embodiments of the present method provide for the automatic identification of an optimal image frame that allows for efficient measurement of one or more biometric parameters of the target structure having a defined geometrical shape. For clarity, the present method is described with reference to detection and identification of an elliptical head region in ultrasound image frames corresponding to a fetus. However, it may be appreciated that other anatomical structures may similarly be identified using embodiments of the present method.

The method begins at step 202, where a plurality of 3D image frames corresponding to a VOI in a subject, for example, a fetus may be received. In one embodiment, the image frames may be received from an acquisition subsystem, such as the ultrasound system 100 of FIG. 1, which may be configured to acquire imaging data corresponding to the VOL Additionally, position information may be received from a position sensor such as the position sensor 113 of FIG. 1. In an alternative embodiment, however, the 3D image frames and the position information may be received from a storage repository such as the memory device 118 of FIG. 1 that may be configured to store the position information and the previously acquired images of the fetus.

It may be desirable to determine a presence of one or more target structures in the plurality of image frames for use in clinical diagnosis. In certain embodiments, the target structure may be detected in an image frame by applying a shape fitting algorithm to a given a set of 3D points that define a boundary of the target structure. Specifically, in certain embodiments, the target structure may be identified from 3D points corresponding to one or more candidate structures present in each image frame.

Accordingly, at step 204, a set of candidate structures may be identified in each image frame in the plurality of 3D image frames. The candidate structures, for example, may be identified using gray scale morphology, Otsu thresholding, a geometrical statistics based classification, and/or any other suitable structure identification method.

Further, at step 206, a plurality of edge points corresponding to a set of candidate structures in each image frame in the plurality of 3D image frames may be determined In one embodiment, the edge points may be determined by applying an edge detection function to different imaging planes along a specific coordinate axis in each image frame. By way of example, in one embodiment, a 3D VOI may be represented as a product (M×N×L), where M corresponds to a width, N corresponds to a height, and L corresponds to a length of the VOL In such an embodiment, a cloud of 3D edge points in the 3D volume may be determined by applying, for example, a 2D canny edge detector on L scan planes of size (M×N) along the z-axis. However, in other embodiments, the edge detector may be applied to different axes to generate different sets of 3D edge points.

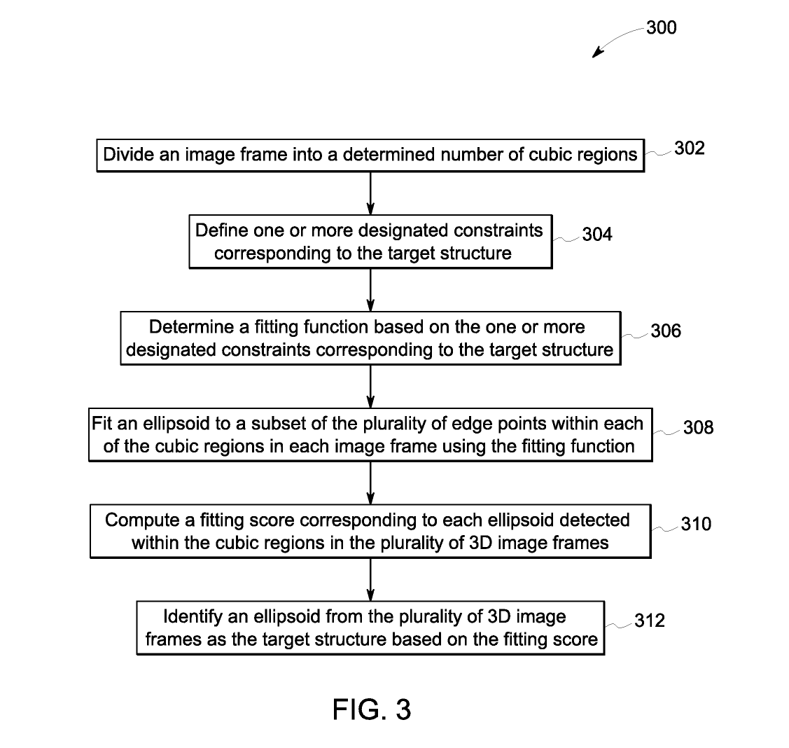

Further, at step 208, a target structure may be identified from the set of candidate structures. In one embodiment, a constrained shape fitting may be applied to the plurality of edge points in each image frame in the plurality of 3D image frames to identify the target structure. An embodiment of constrained shape fitting for use in identifying the target structure in accordance with aspects of the present specification will be described in greater detail with reference to FIG. 3.

Referring now to FIG. 3, a flow chart 300 illustrating an exemplary method for detecting a target structure in an image frame using constrained shape fitting is depicted. The method begins at step 302, where an image frame may be divided into a determined number of cubic regions. In one embodiment, the determined number may depend upon a size of the image frame, user input, and/or application requirements. In one embodiment, for example, the image frame may be divided into 1000 cubic regions (10×10×10), which are evenly spaced in the 3D VOL Each cubic region includes a corresponding set of 3D edge points, including the3D edge points corresponding to the candidate structures, which may be used in detecting a boundary of the target structure in the image frame.

In certain embodiments, the target structure may be defined as a 3D conic structure using a second order polynomial equation. One example of such a polynomial is provided in equation (1).

F(a, x)=a·x (1)

a·x=ax²+bxy+cy²+dxz+eyz+fz² +gx+hy+kz+l=0 (2)

In equations (1) and (2):

x=[x², xy, y², xz, yz, z², x, y, z, 1]^{T} (3)

a=[a, b, c, d, e, f, g, h, i, j, k, l]^{T} (4)

where x corresponds to a location vector representative of location coordinates corresponding to each of the 3D edge points, a corresponds to a set of shape parameters corresponding to the target structure, and T corresponds to a transpose function.

As previously noted with reference to step 208 of FIG. 2, constrained shape fitting may be used to identify the desired 3D conic structure corresponding to the target structure in the image frame. As the shape of a fetal head resembles an ellipsoid, in the present embodiment, the constrained shape fitting may correspond to an ellipsoid fitting method. Furthermore, for computational efficiency, the ellipsoid fitting method may restrict a search space to only candidate structures that approximate a known shape of the target structure, for example, a fetal head.

Thus, at step 304, one or more designated constraints corresponding to the target structure may be defined. In one embodiment, the constraints may be defined such that the 3D conic structure, for example, represented using equations (1) and (2), approximates a shape of the fetal head. Accordingly, certain geometrical constraints may be applied to the shape parameters in equation (1) such that the 3D conic structure corresponds to an ellipsoid. An example of the constraints imposed on the shape parameters may be represented using equation (5).

f_{c1}(a)=4ac−b²>0 (5)

where f_{c1}(a) corresponds to an exemplary constraint and a, b and c are representative of shape parameters corresponding to the desired 3D conic structure.

Additionally, in view of a known geometry of typical fetal heads, it may be assumed that the fetal head may not be long or flat. Accordingly, a ratio of long axis to short axis of an ellipsoid representative of the fetal head may be a non-zero value that is greater than one but close to one. Thus, an additional constraint for minimizing the ratio of the long axis to the short axis of the ellipsoid may be imposed on equation (1). An example of such an additional constraint f_{c2}(a) may be represented using equation (6).

f_{c2}(a)=2a²+2c²+2f²+b²+d²+e²−2ac−2af−2cf (6)

where f_{cf}(a) is minimized

In accordance with certain other aspects of the present specification, the first and second constraint functions defined in equations (5) and (6) may alternatively be represented using equations (7) and (8), and (9) and (10), respectively.

$$\begin{matrix}{f}_{c 1} ( a ) = {a}^{T} {C}_{1} a & ( 7 ) \\ {C}_{1}^{9 \times 9} = ( \begin{matrix}0 & 0 & 2 & 0 \\ 0 & - 1 & 0 & 0 \\ 2 & 0 & 0 & 0 \\ 0 & 0 & 0 & {0}^{6 \times 6}\end{matrix} ) & ( 8 ) \\ {f}_{c 2} ( a ) = {a}^{T} {C}_{2} a & ( 9 ) \\ {C}_{1}^{9 \times 9} = ( \begin{matrix}2 & 0 & - 1 & 0 & 0 & - 1 & 0 \\ 0 & 1 & 0 & 0 & 0 & 0 & 0 \\ - 1 & 0 & 2 & 0 & 0 & - 1 & 0 \\ 0 & 0 & 0 & 1 & 0 & 0 & 0 \\ 0 & 0 & 0 & 0 & 1 & 0 & 0 \\ - 1 & 0 & - 1 & 0 & 0 & 2 & 0 \\ 0 & 0 & 0 & 0 & 0 & 0 & {0}^{3 \times 3}\end{matrix} ) & ( 10 )\end{matrix}$$

where C₁ and C₂ correspond to first and second constraint matrices.

Although the present embodiment describes an ellipsoid fitting method with suitable constraints for detecting a fetal head, in alternative embodiments, other shape fitting methods employing suitable fitting functions and geometrical constraints may be used to fit target structures of different shapes.

Further, at step 306, a fitting function based on the one or more designated constraints corresponding to the target structure may be determined In a presently contemplated embodiment, a suitable fitting function for fitting an ellipsoid to N number of 3D edge points may be determined using equations (1), (2), (8), and (10). The fitting function may be represented, for example, using equation (11).

$$\hat{a} = arg {min}_{a} {\sum }_{i = 1}^{N} F ( a , {x}_{i} ) + γ N \cdot {a}^{T} {C}_{2} a = arg {min}_{a} {a}^{T} ( {D}^{T} D + γ {NC}_{2} ) a \\ subject to \\ {a}^{T} {C}_{1} a > 0 \qquad ( 11 )$$

where D=(x₁, x₂ . . . , x_{N})^{T} corresponds to a matrix composed of N 3D edge points, xᵢ corresponds to a location vector of an edge point such as represented using equation (3), γ corresponds to a determined strength of the second constraint C₁^{9×9}, for example, defined using equation (10), â corresponds to a generalized eigenvector of ((D^{T}D+γNC₂)a=λC₁a), where λ corresponds to a generalized eigenvalue.

Moreover, at step 308, an ellipsoid may be fit to a subset of the plurality of edge points within each of the cubic regions in each image frame using the fitting function. In one embodiment, the fitting function may fit an ellipsoid to the 3D edge points corresponding to a candidate structure in the image frame by solving a constrained optimization problem. The constrained optimization problem, in turn may be solved, for example, using a Lagrange multiplier. In one example, the fitting function may attempt to fit an ellipsoid to the 3D edge points inside each cubic region using the Lagrange multiplier. It may be noted that even though a single cubic region may only include a part of an ellipsoid, the fitting function defined in equation (11) may still be able to estimate one or more ellipsoid parameters. For example, the fitting function may be able to estimate the center of the ellipsoid or length of axes of the ellipsoid in one or more directions. However, only the cubic region that actually includes a part of the fetal head that is typically elliptical will result in a good fit.

Accordingly, at step 310, a fitting score corresponding to each ellipsoid detected within the cubic regions in the plurality of 3D image frames may be computed. In one example, the fitting score E(a) may be computed using equation (12).

$$E ( a ) = exp [ - \frac{1}{N} {\sum }_{i = 1}^{N} F ( a , {x}_{i} ) - γ \cdot {a}^{T} {C}_{2} a ] \qquad ( 12 )$$

Further, at step 312, an ellipsoid may be identified from the plurality of 3D image frames as the target structure based on the fitting score. In one embodiment, an ellipsoid having the highest fitting score may be identified as the fetal head. Alternatively, the ellipsoid having a fitting score greater than a determined threshold may be may be identified as the fetal head. In certain other embodiments, the ellipsoid having a fitting score within a user and/or application designated range may be identified as the fetal head in the image frame being processed.

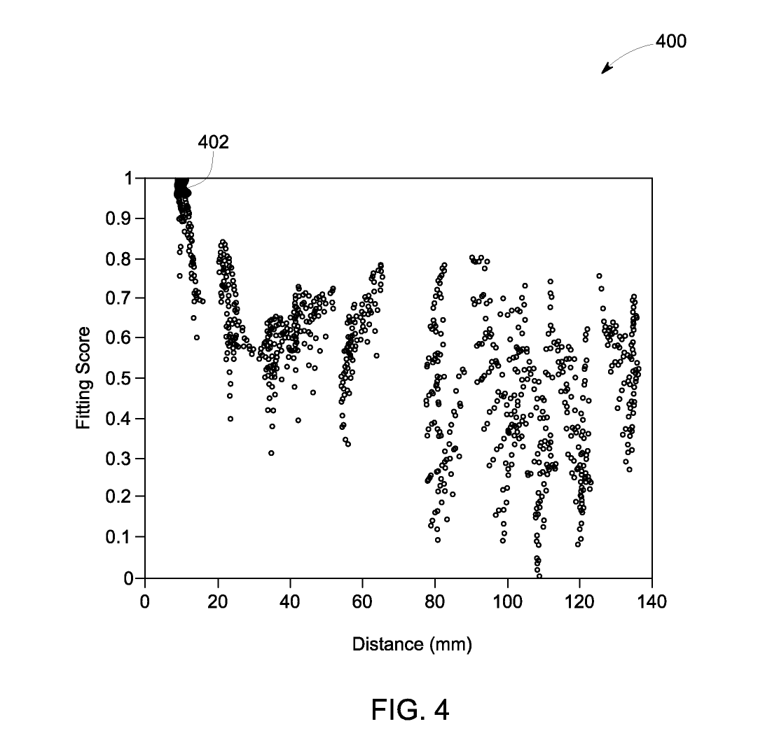

FIG. 4 illustrates a graphical representation 400 of fitting scores computed for a plurality of ellipsoids that are representative of candidate structures detected in an image frame. In the graphical representation 400, the x-axis corresponds to a distance (in millimeters (mm)) of a center of each ellipsoid from a true center of a fetal head and the y-axis corresponds to the fitting score. In one embodiment, the true center of the fetal head may be determined based on manual markings by an experienced and/or skilled radiologist on one or more of the image frames. As is evident from the depictions of FIG. 4, the ellipsoids that are detected in a region proximal the true center of the fetal head have high fitting scores 402, thus indicating the accuracy of the present method for automatically detecting the target structure.

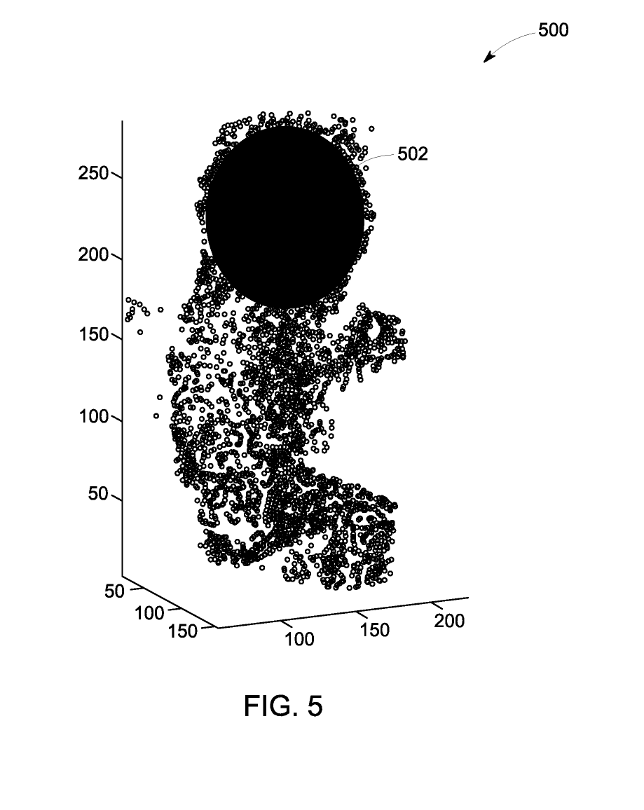

Further, FIG. 5 illustrates a graphical representation 500 of fitting scores computed for a plurality of ellipsoids representative of candidate structures in a plurality of image frames corresponding to different regions of a fetus. In FIG. 5, reference numeral 502 is used to indicate a true head region of a fetus. In one embodiment, the true head region 502 is determined based on manual markings on the image frames by a skilled radiologist. As is evident from the depictions of FIG. 5, the ellipsoids that are detected in the true head region have high fitting scores. Thus, embodiments of the method described with reference to FIG. 3 may be used for detecting the target structure with greater accuracy.

With returning reference to FIG. 2, at step 210, a subgroup of image frames that include the identified target structure may be identified from the plurality of 3D image frames. The edge points in the head region may form an ellipsoid shape, and thus, have higher fitting scores. Accordingly, the subgroup of image frames may typically include the image frames that correspond to a head region of the fetus. Additionally, at step 212, a subset of 3D edge points that correspond to the target structure identified in each image frame in the subgroup of image frames may also be identified.

Once the target structure and corresponding 3D edge points are identified in each image frame in the subgroup of image frames, one or more desired biometric measurements may be determined using at least one of the subgroup of image frames. Accurate measurements of biometric parameters, however, entail identification of a clinically prescribed scan plane from a plurality of candidate scan planes that may be defined using the subset of 3D edge points corresponding to the target structure.

Accordingly, at step 214, a plurality of 2D candidate scan planes corresponding to the subset of the edge points in each of the subgroup of image frames may be identified. As previously noted, a desired scan plane for accurate BPD and HC measurements includes a cavum septum pellucidum, thalami and choroid plexus in the atrium of lateral ventricles such that the cavum septum pellucidum appears as an empty box and the thalami resemble a butterfly.

Conventionally, a binary classifier is used to classify clinically suitable and unsuitable scan planes for BPD and HC measurements. For example, one classifier may employ an Active Appearance Model (AAM) and Linear Discriminative Analysis (LDA) to assign a positive score to a suitable scan plane and a negative score to an unsuitable scan plane. However, as the classifier may only focus on discriminating between the suitable and unsuitable scan planes, scan planes that are close to a suitable scan plane and far from an unsuitable scan plane may also have positive scores. Selecting the correct scan plane from multiple scan planes with comparable positive scores adds further complications to a clinical diagnosis.

Accordingly, embodiments described herein provide an exemplary method that may be adapted to identify a clinically prescribed or desired scan plane for making one or more desired measurements corresponding to one or more target structures. Particularly, embodiments of the present method allow for identification of a desired 2D scan plane from a 3D VOI for making the desired biometric measurements.

In one embodiment, a search for the desired scan plane may be initialized based on the 3D edge points that correspond to the target structure, for example, an ellipsoid identified as the fetal head in step 208. Moreover, a 2D scan plane that crosses the center of the ellipsoid and is perpendicular to the long axis of the ellipsoid may be selected as an “initial” scan plane.

Generally, a 2D scan plane position may be represented using equation (13).

p=(φ, θ, ψ, Cₓ, C_{y}, C_{z}) (13)

where (φ, θ, ψ) correspond to rotation parameters and (Cₓ, C_{y}, C_{z}) correspond to translation parameters with respect to x-axis, y-axis, and z-axis, respectively.

Accordingly, in one embodiment, for a given a scan plane position, a 2D image I may be extracted from a 3D VOI, V. Such a 2D image I, for example, may be represented using equation (14).

I=V(W(p)) (14)

where W(p) corresponds to 3D edge points located on the 2D scan plane.

The 3D edge points W(p), in turn, may be computed by translating 2D coordinates (x, y)^{T} to a 3D space, for example, using equation (15).

$$W ( p ) = ( \begin{matrix}{W}_{x} \\ {W}_{y} \\ {W}_{z}\end{matrix} ) = λ ( \begin{matrix}cos θ cos ψ & - cos φ sin ψ + sin φ sin θcos ψ & {C}_{x} \\ cos θ sin ψ & - cos φ sin ψ + sin φ sin θsinψ & {C}_{y} \\ - sin θ & sin φ cos θ & {C}_{z}\end{matrix} ) ( \begin{matrix}x \\ y \\ 1\end{matrix} ) \qquad ( 15 )$$

where λ is a scale factor.

Thus, equations (14) and (15) may be used to identify the plurality of 2D candidate scan planes from the 3D VOI.

Further, at step 216, the plurality of 2D candidate scan planes may be ranked using a determined ranking function. In one embodiment, the determined ranking function may correspond to a Boosted Ranking Model (BRM). Typically, a BRM corresponds to a classification methodology that applies sequentially reweighted versions of input data to a classification algorithm, and determines a weighted majority of sequence classifiers, thus produced. At each application of the classification algorithm to the reweighted input data, the classification algorithm learns an additional classifier that corrects errors made by the weighted majority of the previously learned classifiers.

Accordingly, given a pair of ranked training image frames (x¹, x²), a presently contemplated embodiment of the BRM may be used to learn or train a ranking function F(x) such that F(x¹)>F(x²) if x¹ is ranked higher than x² based on a designated criterion. However, if x² is ranked higher than x¹, the BRM may be used to learn a ranking function F(x) such that F(x¹)≦F(x²). In one embodiment, the BRM may employ a plurality of weak rankers that focus on different sub-parts of an image frame for obtaining classification information. As used herein, the term “weak ranker” may be used to refer to a classifier that provides an error rate that is better than a random rate of error. In one embodiment, such a weak ranker may be represented, for example, using equation (16).

$${f}_{t} ( x ) = \frac{1}{π} arctan ( {g}_{t} {x}_{t} - {b}_{t} ) \qquad ( 16 )$$

where xₜ corresponds to one dimension of the data vector x, gₜ ∈ {−1, +1} is indicative of a sign (positive or negative) of a decision function, and bₜ corresponds to a determined threshold.

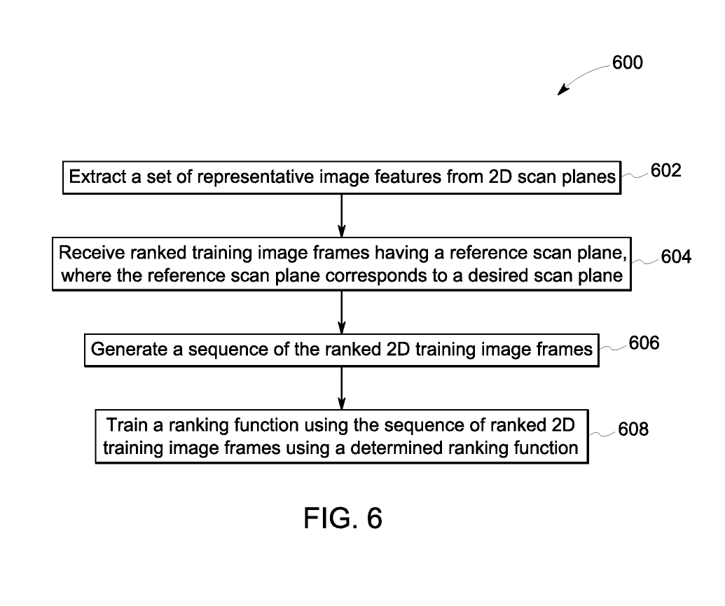

Subsequently, the plurality of weak rankers may be combined to determine the ranking function F(x), which when adequately trained, provides enhanced classification performance. An embodiment of a method for training a ranking function F(x) will be described in greater detail with reference to FIG. 6.

FIG. 6 depicts a flow chart 600 illustrating an exemplary method for training a ranking function for use in accurately ranking a plurality of 2D candidate scan planes. In particular, the method of FIG. 6 corresponds to the step 216 of FIG. 2. The method beings at step 602, where a set of representative image features such as color and/or intensity may be extracted from the 2D candidate scan planes. In one embodiment, the image features may be extracted, for example, using MRP that allows for a computationally efficient detection of a desired scan plane.

Accordingly, given a 2D scan plane, MRP may be used to project a 2D scan plane image to a lower dimensional space where clinically suitable scan planes are easily distinguishable from clinically unsuitable scan planes. In one embodiment, for example, a 2D scan plane of size 75 mm×75 mm may be extracted from a 3D VOL Subsequently, the 2D scan plane may be resized to a 2D image having a size of about 25×25 pixels. A corresponding scan plane image I, thus, may be represented as a 625 (25×25) dimensional vector. In accordance with aspects of the present specification, MRP may be used to project the scan plane image I to a lower dimension, for example, to a 200 dimensional data vector x. In one embodiment, the scan plane image I may be projected to the data vector x, for example, using equation (17).

x=M^{T}I (17)

where M^{T} corresponds to a projection matrix used to project the scan plane image I to a determined lower dimensional data space.

Further, at step 604, training image frames having a reference scan plane may be received, where the reference scan plane corresponds to a desired scan plane. In one embodiment, pairs of 2D ultrasound (I_{2D}) and 3D image frames may be received as the training image frames. Each of the 2D and 3D training image frames may include manual markings by a skilled radiologist to indicate the reference scan plane. As used herein, the term “reference scan plane” may be used to refer to the desired and/or clinically acceptable scan plane that is prescribed for obtaining biometric measurements of interest. Additionally, in certain embodiments, the radiologist may also rank the 2D and 3D training image frames based on a suitability of the image frame for use in the desired biometric measurements.

Moreover, at step 606, a sequence of ranked 2D training image frames may be generated. For example, given manually labeled scan plane position p* in a 3D volumetric image, a sequence of 2D images may be generated by uniformly adding perturbations to the manually marked reference scan plane position, p*. An exemplary sequence, thus generated, may be represented using equation (18).

p*:{p*+νΔp}_{ν=0, . . . V} (18)

where Δp corresponds to a unit perturbation per rotation parameter and/or translation parameter and ν corresponds to a magnitude of perturbation.

In one embodiment, the ranked 2D training image frame sequence may be generated by adding determined perturbations, for example, of zero, four, eight, twelve, sixteen, and twenty degrees to the manually marked reference scan plane position, p*. The determined perturbations, in one example, may be represented using equations (19) and (20).

Δp=(4, 0, 0, 0, 0, 0)^{T} (19)

ν=0, 1, 2, 3, 4, 5 (20)

Moreover, in one embodiment, a ranked training image frame pair may be represented, for example, using equation (21).

Ranked pair=(V(W(p*+νΔp)), V(W(p*+(ν+1)Δp))) (21)

As the 2D training image frames I_{2D} received at step 604 are manually selected by the radiologist as being representative of clinically acceptable image frames, typically, the scan planes corresponding to the 2D training image frames I_{2D} may be ranked the same or higher than scan planes corresponding to the 3D volumetric images. Accordingly, a pair of ranked training image frames may also be alternatively represented, for example, using equation (22).

Ranked pair=(I_{2D}, V(W(p*+νΔp)))_{ν>0} (22)

Further, at step 608, the sequence of ranked 2D training image frames may be used to train a ranking function F(x) using a determined ranking function, for example, a method employing a BRM. An exemplary implementation of a BRM for training the ranking function given a set of ranked training pairs (xᵢ¹, xᵢ²)_{i=1 . . . N} is depicted in the present specification using Algorithm 1.

```
Algorithm 1
Input: Training data pairs (xᵢ¹, xᵢ²)_{i=1...N} and their labels yᵢ = +1 if xᵢ¹ ranked
higher than xᵢ², else yᵢ = −1
$Initialize weight of data pairs \text{:} {w}_{i} = \frac{1}{N}$
for t = 1 to T do
  Fit a weak ranker fₜ to minimize the least square error:
    fₜ = arg min Σi wᵢ[yᵢ − hₜ(xᵢ¹, xᵢ²)]² (23)
  where hₜ (xᵢ¹, xᵢ²) = fₜ(xᵢ¹) − fₜ(xᵢ²)
Update weights wᵢ ← wᵢe^{-y}^{ₜ}^{h}^{ₜ}^{(x}^{ᵢ}^{¹}^{,x}^{ᵢ}^{²}⁾
Normal weights such as Σᵢ wᵢ = 1
end for
return
```

A strong ranker may then be determined using a linear combination of weak rankers, as represented by equation (24).

F(x=Σₜfₜ(x) (24)

In Algorithm 1, hₜ(xᵢ¹, xᵢ²) corresponds to a weak classifier that includes outputs of a weak ranker fₜ(x) corresponding to two data samples xᵢ¹ and xᵢ². When xᵢ¹ is ranked higher than xᵢ², the weak classifier hₜ(xᵢ¹, xᵢ²) is determined to be closer to +1, thus indicating a scan plane closer to the desired scan plane. However, a value of the weak classifier hₜ(xᵢ¹, xᵢ²) that is closer to −1 is considered to be indicative of a clinically unsuitable scan plane. As previously noted, a combination of the weak rankers may be used to determine the ranking function F(x). The ranking function F(x), in turn, may be used to assess a suitability of a 2D candidate scan plane for making accurate measurements of biometric parameters corresponding to the target structure.

In one example, the ranking function F(x) may be determined based on equations (14), (17), and (24). Particularly, a ranking function F(p) for a particular 2D candidate scan plane p may be represented, for example, using equation (25).

$$F ( p ) = \frac{1}{π} {\sum }_{t = 1}^{T} ( {g}_{t} {M}_{t}^{T} V ( W ( p ) ) - {b}_{t} ) \qquad ( 25 )$$

where gₜ corresponds to the sign of the decision function defined in equation (16), Mₜ^{T} corresponds to the projected matrix defined in equation (17), V corresponds to 3D volumetric data, and bₜ corresponds to a threshold of the decision function defined in equation (16).

Furthermore, according to exemplary aspects of the present specification, the ranking function F(p) may be employed to iteratively assess and/or rank different 2D candidate scan planes. In one embodiment, the iterative ranking may be implemented using a gradient ascent search. For example, if a 2D candidate scan plane at the i^{th} iteration is represented as pⁱ, the 2D candidate scan plane in subsequent iterations may be represented, for example, using equation (26).

$${p}^{i + 1} = {p}^{i} + κ \frac{\partial F}{\partial p} \qquad ( 26 )$$

where κ corresponds to a suitable constant.

In certain embodiments, a gradient

$$\frac{\partial F}{\partial p}$$

of the ranking function F(p) may be determined for updating scan plane locations defined in equation (13), for example, using equation (27).

$$\frac{\partial F}{\partial p} = \frac{1}{π} {\sum }_{t = 1}^{T} \frac{{g}_{t} {M}_{t}^{T} Δ V \frac{\partial W}{\partial p}}{1 + {( {g}_{t} {M}_{t}^{T} V ( W ( p ) ) - {b}_{t} )}^{2}} \qquad ( 27 )$$

where ΔV corresponds to a gradient of the 3D VOI W(p) corresponding to equation (14) and

$$\frac{\partial W}{\partial p}$$

corresponds to a Jacobian of the 3D coordinates W(p) of equation (15).

Thus, in one embodiment, each of the 2D candidate scan planes may be ranked using the ranking function F(p). In particular, each of the 2D candidate scan planes may be ranked using equations (25), (26), and (27).

With returning reference to FIG. 2, following the processing of steps 202-216, a plurality of ranked 2D candidate scan planes may be generated. Subsequently, at step 218, a desired scan plane may be identified from the plurality of ranked 2D candidate scan planes in the plurality of 3D image frames based on the ranking. In one embodiment, if the scan plane pⁱ in equation (26) converges at a particular iteration, the corresponding candidate scan plane may be identified as the desired scan plane. Alternatively, the 2D candidate scan plane having the highest or lowest rank may be identified as the desired scan plane. Additionally, an image frame that includes the desired scan plane may be identified from the subgroup of image frames, as indicated by step 220. Furthermore, in certain embodiments, the selected image frame may be automatically frozen to allow for further processing.

Moreover, at step 222, a diagnostic parameter corresponding to the target structure may be measured using the selected image frame. The diagnostic parameter, for example, may include a BPD or an HC of a fetus. Accordingly, in one embodiment, automated measurements of the BPD and HC of a fetus may be triggered when the image frame that includes a visualization of the fetal head region in the desired scan plane is identified. Use of the boosted ranking function, thus, allow for automatic identification of clinically acceptable scan planes for providing robust and reproducible measurements of biometric parameters corresponding to the target structure in real-time.

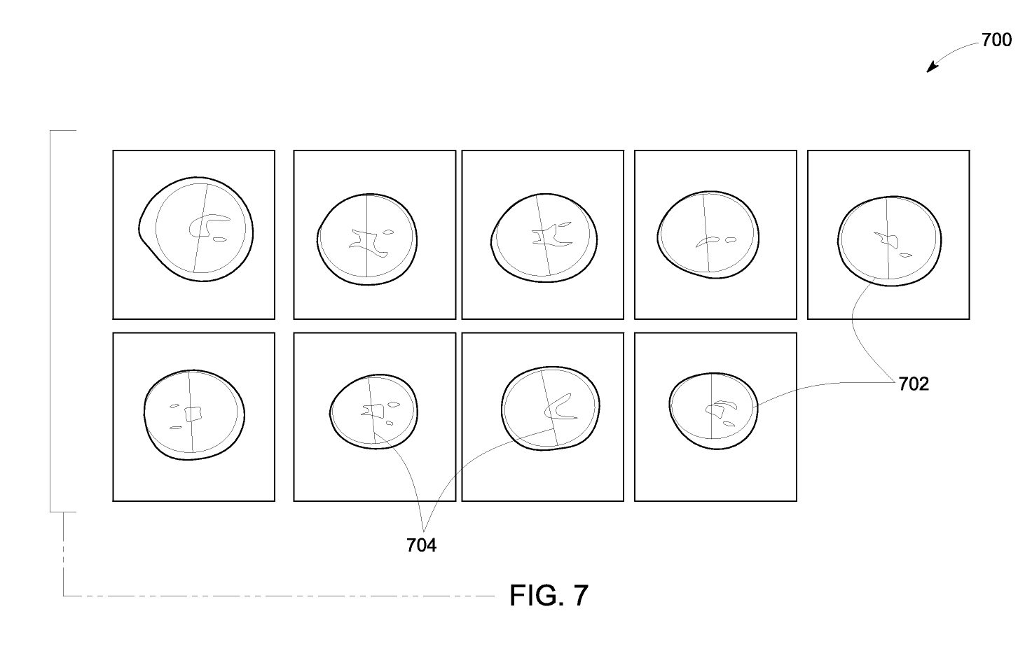

FIG. 7 depicts a diagrammatical representation 700 of a plurality of image frames of a VOI in a subject. Particularly, in the embodiment illustrated in FIG. 7, the VOI corresponds to a head region of a fetus. The head region is detected using an embodiment of the ellipsoid fitting method described with reference to FIGS. 2 and 3. Further, the scan planes corresponding to each image frame are ranked using the boosted ranking function, such as the BRM described with reference to FIG. 6. Subsequently, a scan plane having the highest rank is selected as the desired scan plane. As illustrated in FIG. 7, the desired scan planes identified by embodiments of the present methods in a majority of the image frames include clear butterfly-like structures indicative of a clinically prescribed position and orientation of the thalami. Accordingly, BPD and HC are automatically measured using the image frame that included the fetal head in the desired/clinically prescribed scan plane.

A performance evaluation of an embodiment of the boosted ranking method is presented in the present specification with reference to FIG. 7. Specifically, HC and BPD measurements obtained from a head region that is automatically detected in a desired scan plane using an embodiment of the present method are compared to measurements obtained from regions in image frames that are manually labeled by a skilled radiologist to indicate a location of a true HC 702 and true BPD 704.

Table 1 presents HC and BPD measurements made using the desired scan plane identified using an embodiment of FIG. 6.

TABLE 1

| Image | 1 | 2 | 3 | 4 | 5 | 6 | 7 | 8 | 9 |
|---|---|---|---|---|---|---|---|---|---|
| HC | 178.8 | 179.5 | 184.0 | 179.9 | 183.3 | 182.0 | 173.5 | 176.5 | 189.1 |
| (mm) | | | | | | | | | |
| BPD | 55.5 | 55.4 | 56.8 | 55.4 | 56.8 | 57.6 | 57.1 | 54.3 | 56.4 |
| (mm) | | | | | | | | | |

It may be noted that the clinically prescribed values for true HC and BPD measurements correspond to 180 mm and 52 mm, respectively. An average error in HC and BPD measurements may be determined by computing an average of a difference between the true HC and BPD measurements (180 mm and 52 mm, respectively) and each of the corresponding measurements listed in Table 1. Accordingly, the average error of HC and BPD measurement using the embodiments of the methods described in the present specification are determined to have a value of about 3.4 mm and 4.1 mm, respectively.

Embodiments of the present specification, thus, provide systems and methods that allow for automated detection of the target structure, identification of the clinically prescribed scan plane, and measurement of the diagnostic parameters corresponding to the target structure in real-time. Such an automated and real-time detection of the desired scan plane eliminates subjective manual assessment of the ultrasound images. Moreover, embodiments of the present specification allow for a reduction in imaging time, while providing enhanced performance over conventional learning and segmentation based methods.

Particularly, the automated identification of the optimal image frame allows for robust and reproducible measurements of the target structure irrespective of the skill and/or experience level of the user. The embodiments of the present methods and systems, thus, aid in extending quality ultrasound imaging services over large geographical areas including rural regions that are traditionally under-served owing to lack of trained radiologists.

Furthermore, use of a position sensor such as the position sensor 113 of FIG. 1 may provide additional information that may be used for reconstruction of high quality 3D ultrasound images without use of expensive 3D ultrasound probes. The position sensors are suitable for use with most 2D ultrasound probes and may be fitted unobtrusively to allow scanning of large volumes of subject. Additionally, as these position sensors are typically inexpensive as compared to the 3D ultrasound probes, use of the position sensors in addition to efficient image reconstruction methods may allow for manufacture of cost-effective yet high quality ultrasound imaging systems. Although the present specification describes a configuration of an ultrasound system including the position sensor, it may be noted that in an alternative embodiment, an ultrasound system may be configured to implement the present method without use on any position sensors.

Additionally, certain embodiments of the present methods and systems may also allow for an objective assessment of performance of different imaging systems, positions sensors, image reconstruction algorithms, and/or effect of operator variability on the biometric measurements. The objective assessment may be based on a comparison of a measured value of a diagnostic parameter obtained by the different configurations employed for imaging the subject. For example, embodiments of the present methods may be used to compare imaging systems that include different position sensors, imaging systems devoid of position sensors, efficiency of different operators, and/or image reconstruction algorithms. Specifically, the comparison may be made based on measurements obtained by the different systems, position sensors, operators and/or image reconstruction methods on the same set of images with a reference measurement obtained through manual markings by a skilled radiologist. Embodiments of the present methods and systems, thus may aid in selection of a combination of suitable systems, position sensors, and/or imaging methods that provide greater accuracy, efficiency, and/or cost-effectiveness.

It may be noted that the foregoing examples, demonstrations, and process steps that may be performed by certain components of the present systems, for example by the processing unit 114 of FIG. 1, may be implemented by suitable code on a processor-based system. The processor-based system, for example, may include a general-purpose or a special-purpose computer. It may also be noted that different implementations of the present specification may perform some or all of the steps described herein in different orders or substantially concurrently.

Additionally, the functions may be implemented in a variety of programming languages, including but not limited to Ruby, Hypertext Preprocessor (PHP), Perl, Delphi, Python, C, C++, or Java. Such code may be stored or adapted for storage on one or more tangible, machine-readable media, such as on data repository chips, local or remote hard disks, optical disks (that is, CDs or DVDs), solid-state drives, or other media, which may be accessed by the processor-based system to execute the stored code.

Although specific features of embodiments of the present specification may be shown in and/or described with respect to some drawings and not in others, this is for convenience only. It is to be understood that the described features, structures, and/or characteristics, illustrated in the figures and described herein, may be combined and/or used interchangeably in any suitable manner in the various embodiments, for example, to construct additional assemblies and methods for use in diagnostic imaging.

While only certain features of the present specification have been illustrated and described herein, many modifications and changes will occur to those skilled in the art. It is, therefore, to be understood that the appended claims are intended to cover all such modifications and changes as fall within the true spirit of the invention.

1. A system for imaging a subject, comprising:
an acquisition subsystem configured to obtain a plurality of three-dimensional image frames corresponding to a volume of interest in the subject;
a processing unit in operative association with the acquisition subsystem and configured to:
determine a plurality of edge points corresponding to a set of candidate structures in each image frame in the plurality of three-dimensional image frames;
identify a target structure from the set of candidate structures by applying constrained shape fitting to the plurality of edge points in each image frame in the plurality of three-dimensional image frames;
identify a subgroup of image frames from the plurality of three-dimensional image frames, wherein each image frame in the subgroup of image frames comprises the target structure;
determine a subset of edge points corresponding to the target structure from the plurality of edge points in each image frame in the subgroup of image frames;
determine a plurality of two-dimensional candidate scan planes corresponding to the subset of edge points in each image frame in the subgroup of image frames;
rank the plurality of two-dimensional candidate scan planes corresponding to each image frame in the subgroup of image frames using a determined ranking function;
identify a desired scan plane from the plurality of two-dimensional candidate scan planes based on the ranking; and
measure a diagnostic parameter corresponding to the target structure using a selected image frame in the plurality of three-dimensional image frames, wherein the selected image frame comprises the desired scan plane.

2. The system of claim 1, wherein the system is an ultrasound imaging system, a contrast enhanced ultrasound imaging system, an optical imaging system, or combinations thereof.

3. The system of claim 1, wherein the acquisition subsystem comprises an imaging probe configured to acquire image data corresponding to the volume of interest in the subject.

4. The system of claim 3, wherein the acquisition subsystem further comprises a position sensor operationally coupled to the imaging probe and configured to determine position information corresponding to the imaging probe.

5. The system of claim 4, wherein the position sensor comprises an acoustic sensor, an electromagnetic sensor, an optical sensor, an inertial sensor, a magnetoresistance sensor, or combinations thereof.

6. The system of claim 1, wherein the processing unit is configured to:
identify a plurality of desired scan planes corresponding to a plurality of optimal image frames generated by two or more imaging systems, image reconstruction algorithms, or a combination thereof, using the ranking function;
measure a value of the diagnostic parameter using each of the plurality of desired scan planes corresponding to the plurality of optimal image frames;
compare the measured value of the diagnostic parameter with a reference value of the diagnostic parameter;
assess performance of the two or more imaging systems, the image reconstruction algorithms, or a combination thereof, based on the comparison of the measured value and the reference value of the diagnostic parameter; and
output the assessed performance via an output device operatively coupled to the processing unit.

7. The system of claim 1, further comprising a display device operatively associated with the processing unit, wherein the display device is configured to display the plurality of three-dimensional image frames, the desired scan plane, the selected image frame, one or more measurements corresponding to the diagnostic parameter, or combinations thereof.

8. A method for ultrasound imaging of a subject, comprising:
determining a plurality of edge points corresponding to a set of candidate structures in each image frame in a plurality of three-dimensional image frames corresponding to a volume of interest in the subject;
detecting a target structure from the set of candidate structures by applying constrained shape fitting to the plurality of edge points in each image frame in the plurality of three-dimensional image frames;
identifying a subgroup of image frames from the plurality of three-dimensional image frames, wherein each image frame in the subgroup of image frames comprises the target structure;
determining a subset of edge points corresponding to the target structure from the plurality of edge points in each image frame in the subgroup of image frames;
determining a plurality of two-dimensional candidate scan planes corresponding to the subset of edge points in each image frame in the subgroup of image frames;
ranking the plurality of two-dimensional candidate scan planes corresponding to each image frame in the subgroup of image frames using a determined ranking function;
identifying a desired scan plane from the plurality of two-dimensional candidate scan planes based on the ranking; and
measuring a diagnostic parameter corresponding to the target structure using a selected image frame in the subgroup of image frames, wherein the selected image frame comprises the desired scan plane.

9. The method of claim 8, wherein determining the plurality of edge points corresponding to the set of candidate structures in each image frame comprises applying edge detection to one or more coordinate axes corresponding to each image frame.

10. The method of claim 8, wherein detecting the target structure comprises applying constrained ellipsoid fitting to the plurality of edge points in each image frame in the plurality of three-dimensional image frames.

11. The method of claim 10, wherein applying the constrained ellipsoid fitting comprises:
dividing each image frame into a determined number of cubic regions;
determining a fitting function based on one or more designated constraints corresponding to the target structure;
fitting an ellipsoid to a subset of the plurality of edge points within each of the cubic regions in each image frame using the fitting function;
computing a fitting score corresponding to each ellipsoid detected within each of the cubic regions in the plurality of three-dimensional image frames; and
identifying an ellipsoid from the plurality of three-dimensional image frames as the target structure based on the fitting score.

12. The method of claim 11, further comprising defining the one or more designated constraints, wherein the one or more designated constraints comprise a constraint that a ratio of a long axis to a short axis of the ellipsoid identified as the target structure is minimized

13. The method of claim 12, further comprising identifying a scan plane that crosses a center of the ellipsoid identified as the target structure and is perpendicular to the long axis of the corresponding ellipsoid as an initial scan plane.

14. The method of claim 11, wherein identifying the ellipsoid comprises selecting the ellipsoid having the highest fitting score as the target structure.

15. The method of claim 11, wherein identifying the ellipsoid comprises selecting the ellipsoid having a fitting score greater than a determined threshold as the target structure.

16. The method of claim 8, wherein ranking the plurality of two-dimensional candidate scan planes comprises using a boosted ranking function for identifying the desired scan plane from the plurality of two-dimensional candidate scan planes.

17. The method of claim 8, wherein ranking the plurality of two-dimensional candidate scan planes comprises:
providing a training image frame comprising a reference scan plane, wherein the reference scan plane corresponds to the desired scan plane;
generating a sequence of ranked two-dimensional training image frames by uniformly adding perturbations to the reference scan plane in the training image frame;
training a ranking function using the sequence of ranked two-dimensional training images; and
ranking the two-dimensional candidate scan planes in the plurality of three-dimensional image frames using the ranking function.

18. The method of claim 8, further comprising:
identifying a plurality of desired scan planes corresponding to a plurality of optimal image frames generated by two or more imaging systems, image reconstruction algorithms, or a combination thereof, using the ranking function;
measuring the diagnostic parameter using each of the plurality of desired scan planes corresponding to the plurality of optimal image frames;
comparing a measured value of the diagnostic parameter with a reference value of the diagnostic parameter; and
assessing performance of the two or more imaging systems, the image reconstruction algorithms, or a combination thereof, based on the comparison of the measured value and the reference value of the diagnostic parameter.

19. The method of claim 8, wherein identifying the desired scan plane from the plurality of two-dimensional candidate scan planes in the plurality of three-dimensional image frames comprises performing an iterative gradient descent search using the determined ranking function.

20. The method of claim 8, wherein the diagnostic parameter corresponding to the target structure comprises a biparietal diameter, a head circumference, or a combination thereof, corresponding to a fetus.

21. A non-transitory computer readable medium that stores instructions executable by one or more processors to perform a method for imaging a subject, comprising:
determining a plurality of edge points corresponding to a set of candidate structures in each image frame in a plurality of three-dimensional image frames corresponding to a volume of interest in the subject;
detecting a target structure from the set of candidate structures by applying constrained shape fitting to the plurality of edge points in each image frame in the plurality of three-dimensional image frames;
identifying a subgroup of image frames from the plurality of three-dimensional image frames, wherein each image frame in the subgroup of image frames comprises the target structure;
determining a subset of edge points corresponding to the target structure from the plurality of edge points in each image frame in the subgroup of image frames;
determining a plurality of two-dimensional candidate scan planes corresponding to the subset of edge points in each image frame in the subgroup of image frames;
ranking the plurality of two-dimensional candidate scan planes corresponding to each image frame in the subgroup of image frames using a determined ranking function;
identifying a desired scan plane from the plurality of two-dimensional candidate scan planes based on the ranking; and
measuring a diagnostic parameter corresponding to the target structure using a selected image frame in the plurality of three-dimensional image frames, wherein the selected image frame comprises the desired scan plane.
